(19) United States
(12) Patent Application Publication
(10) Pub. No.: US 2017/0210684 A1
(43) Pub. Date: Jul. 27, 2017

(54) ZEOLITES FOR HYDROCARBON CONVERSION

(71) Applicants: Joern Ilja Siepmann, Hastings, MN (US); Peng Bai, Minneapolis, MN (US); Michael Tsapatsis, Minneapolis, MN (US); Michael W. Deem, West University Place, TX (US)

(72) Inventors: Joern Ilja Siepmann, Hastings, MN (US); Peng Bai, Minneapolis, MN (US); Michael Tsapatsis, Minneapolis, MN (US); Michael W. Deem, West University Place, TX (US)

(21) Appl. No.: 15/327,738

(22) PCT Filed: Jul. 22, 2015
(86) PCT No.: PCT/US15/41544

§ 371 Date: Jan. 20, 2017

Related U.S. Application Data
(60) Provisional application No. 62/027,579, filed on Jul. 22, 2014.

(51) Int. Cl. C07C 5/27; B01J 29/74

(52) U.S. Cl. CPC C07C 5/2775; B01J 29/7461; B01J 29/74

(57) ABSTRACT

A process is provided for the conversion of a hydrocarbon feedstock using catalyst system comprising one or more zeolites. The zeolites have been identified to be capable of selectively catalyzing the hydroisomerization reactions of linear or slightly branched long-chain hydrocarbons. Also provided is a method for the systematic discovery of zeolite framework types that are suitable for such conversion processes, according to a set of criteria: large affinity towards linear alkanes, high adsorption selectivity of linear over branched alkanes, and low adsorption selectivity of linear alkanes of different molecular weights.

### CROSS-REFERENCE TO RELATED APPLICATION

This application claims priority to U.S. Provisional Application Ser. No. 62/027,579, filed on Jul. 22, 2014, the disclosure of which is expressly incorporated herein by reference as if reproduced in its entirety.

### GOVERNMENT FUNDING

This invention was made with government support under DE-FG02-12ER16362 awarded by Department of Energy. The government has certain rights in the invention.

### TECHNICAL FIELD

This invention relates to processes for the conversion of hydrocarbon feedstock using zeolite catalysts, such as for hydroisomerization dewaxing, to produce diesel oils of high cetane number or lubricant base oils of low pour point and high viscosity index.

### BACKGROUND

Middle to heavy distillates from petroleum refineries contain hydrocarbon feedstock for diesels (C₁₂₋₂₀) and lubricant base oils (C₁₈₋₅₀). For applications in automobiles and machineries, the feedstock can be processed in order to improve its cold flow performance (e.g., low pour point and high viscosity index), stability (e.g., oxidation resistance and thermal stability), and, in the case of diesels, combustion quality (e.g., cetane number). This can be achieved by hydrotreating and hydrocracking, which can break down large molecules to branched, smaller alkanes, and remove impurities including sulfur, nitrogen, and metals, as well as the most reactive hydrocarbons such as aromatics and other unsaturated species. However, these hydroprocessing steps have little effect on linear alkanes that contribute to high pour points and low viscosity indices, and these linear alkanes then generally must be removed or converted in subsequent steps.

Linear alkanes were traditionally removed by solvent dewaxing, and later by catalytic cracking dewaxing. Both techniques can reduce the content of waxy alkanes, but at the same time can also lower the product value because of the reduced yield and less valuable cracked species. Dewaxing by solvent or catalytic cracking can be unacceptable for feedstock comprising essentially all linear alkanes, such as paraffinic feedstock derived from the Fischer-Tropsch synthesis.

Crude oil remains the dominant source for transportation fuels and chemical feedstocks. Improving the efficiency of oil refining has the potential to help extend the supply and reduce the cost of current petroleum products. In the 1950s, crystalline zeolites containing sub-2 nm internal pores emerged as shape/size selective sorbents and catalysts, leading to dramatic improvements in numerous processes utilized by the petrochemical industry. For example, zeolites are used to catalyze the conversion of linear long-chain alkanes to slightly branched alkanes of similar molecular weight, with the goal of reducing the pour point and increasing the viscosity index of lubricant oils. Similar transformations can also be desirable for diesel and other fuel oils, in which shorter alkanes are involved. These hydroisomerization reactions depend on a delicate balance between the degree of framework confinement and the size of alkane molecules.

Zeolites play numerous important roles in modern petroleum refineries and have the potential to advance the production of fuels and chemical feedstocks from renewable resources. The performance of a zeolite as a catalyst can depend on its framework structure and the type or location of active sites. To date, 213 framework types have been synthesized and >330,000 thermodynamically accessible zeolite structures have been predicted. Hence, identification of optimal zeolites for a given application from the large number of candidate structures is attractive for accelerating the pace of materials discovery.

A bi-functional catalyst system comprising a high SiO₂/Al₂O₃ ratio, proton-exchanged zeolite and a supported group VIII metal can catalyze the hydroisomerization reactions of naphtha range distillates (C₅₋₁₂) by converting linear alkanes into branched isomers, thereby increasing the octane number of product gasoline. This type of technology is disclosed in U.S. Pat. No. 3,301,917 with rare-earth-metal, proton-exchanged FAU-type zeolites and Pt as the catalyst, which does not prevent cracking. U.S. Pat. Nos. 3,423,568 and 3,673,267 both use Pt and MOR-type zeolites, a catalyst more selective towards isomerization than cracking at low conversions. Similar technology to dewax feedstocks of heavier hydrocarbons is disclosed in U.S. Pat. No. 4,419,220, which uses Pt and BEA-type zeolites to treat C₁₀₊ species. U.S. Pat. Nos. 5,302,279, 5,358,628, and 5,885,438 combine hydroisomerization with other technologies such as solvent extraction and hydrocracking to further improve lubricant base oil quality. U.S. Pat. No. 4,689,138 discloses the application of a new catalyst system consisting of Pt and silicoaluminophosphate (SAPO) zeolites in a dewaxing process with substantially less cracking. The SAPO zeolites can be of the AEL, ATO, or AFO type, and are described in U.S. Pat. No. 4,710,485. Zeolites with high hydroisomerization selectivity continue to be reported, including combinations of zeolites of different framework types, for example, in U.S. Pat. Nos. 5,990,371, 6,051,129, 6,652,735, 6,962,651, and 2007/0029230.

### SUMMARY

Mildly acidic zeolites with high separation factors for linear alkanes over their branched counterparts can be very conducive to reducing cracking and increasing the fraction of branched species in the hydroisomerization product, which can result from a delicate balance between the degree of framework confinement and the size of alkane molecules. These zeolites can repel more highly branched alkane isomers so as to avoid their further cracking.

The present disclosure describes a set of zeolite framework types, defined according to the connectivity of framework atoms that have high selectivity towards hydroisomerization of linear alkanes but whose use in the dewaxing of hydrocarbon feedstock has not been disclosed or suggested before. The framework atoms can be identified by either three-letter codes given to all experimentally synthesized structures by the Structure Commission of the International Zeolite Association (IZA-SC) or seven-digit numbers for predicted structures in the Predicted Crystallography Open Database (PCOD). In brief summary, the present disclosure describes dewaxing processes, which can comprise contacting, under hydroisomerization conditions, a hydrocarbon feedstock with a bi-functional catalyst system comprising one or more mildly acidic zeolites and at least one hydrogenation component. The framework types of the mildly acidic zeolites can be selected to minimize cracking while maximizing hydroisomerization of linear alkanes. The hydrogenation component can include at least one Group VIII, Group VIB, or Group IB metal. The hydrocarbon feedstock can be subject to hydroprocessing prior to dewaxing, and the product base oil can further be hydrofinished and additized.

The present disclosure also describes a method for systematic discovery of zeolite framework types that are suitable for such dewaxing processes, by evaluating each of a plurality of zeolite framework types according to a set of criteria including: large affinity towards linear alkanes, high adsorption selectivity of linear over branched alkanes, and low adsorption selectivity of linear alkanes of different molecular weights.

### BRIEF DESCRIPTION OF THE DRAWINGS

FIG. 1 Hydrocarbon adsorption: (a) Scatter plot of Henrys constant (k_{H,C18}) versus adsorption enthalpy (ΔH_{ads,C18}) and (b) linear-versus-branched selectivity (S_{B0}) versus pore bumpiness (Δd) at infinite dilution for all structures with the color scale indicating the free pore diameter (d_{free}); (c) scatter plot of loading (Q_{C18}) at P=3 MPa versus k_{H,C18} and (d) linear-versus-branched (S_{B}) and long-versus-short (S_{L}) selectivities at P=3 MPa versus those in the low-pressure limit for all structures retained after the first screening step; (e) adsorption characteristics (P_{HC3}=Q_{C18} S_{B3}/S_{L3}, bars) for the top-10 IZA-SC and PCOD structures and for PCOD-S structures with similar selectivities as the IZA-SC structures. The 7-digit PCOD structure identifiers are given in Table 1.

FIG. 2 shows scatter plots for hydrocarbon adsorption. Henrys constant (k_{H,C18}) in mol/kg Pa, row 1) and linear-versus-branched selectivity in the low-pressure limit (S_{B0}, row 2), loading (Q_{C18} in mmol/kg, row 3), linear-versus-branched selectivity (S_{B3}, row 4), long-over-short selectivity (S_{L3}, row 5), and performance score at P=3 MPa (P_{HC3}, row 6) as a function of the diameter of the largest free-roaming sphere (d_{free}, left), the diameter of the largest included sphere along the free sphere path (d_{incl}, middle), and the density of framework T atoms (ρ_{T}, right).

FIG. 3 shows snapshots of representative sorbate configurations obtained for adsorption from a liquid phase containing an equimolar hydrocarbon mixture at T=573 K and P=3 MPa. Views facing (top row) and along (bottom row) the main channel axis are shown for (a) ATO, (b) MTW, and (c) PCOD 8113534. Zeolite frameworks are depicted as gray lines, and C18, C24, C30, (2C17 and 4C17), and 22C16 molecules are labeled as A, B, C, D, and E, respectively.

### DETAILED DESCRIPTION

A hydrocarbon feedstock for lubricant base oils can vary widely in its composition, with the content of waxy linear alkanes ranging from a few percent to over 90% in the case of Fischer-Tropsch-derived stock. Linear long-chain alkanes can crystallize at above room temperature and cause the pour point of the feed mixture to be much higher than most specifications (e.g., 243-263 K). Introducing branches to the alkane molecules can decrease the pour point. On the other hand, excessive branching can lower the viscosity index. In addition, molecules with branching at two nearby carbon atoms and in the center of a chain can be susceptible to cracking. Therefore, a hydroisomerization dewaxing process can be configured to control for the degree of branching. Other aspects about the feedstock can include the presence of impurities, such as unsaturated aliphatics and aromatics, which can have poor viscosity index and can be oxidized to start a series of reactions that can adversely affect the quality of the lubricant oils. These impurities generally need to be removed along with sulfur, nitrogen, and metals via hydrotreating or hydrocracking, or both, prior to hydroisomerization.

As noted above, zeolites have been used to catalyze the conversion of linear long-chain alkanes to slightly branched alkanes of similar molecular weight. However, as further noted above, the hydroisomerization reactions to convert long-chain alkanes depend on a delicate balance between the degree of framework confinement and the size of alkane molecules. Moreover, experimental testing of all existing known zeolites for a given application would be very time and labor intensive, sometimes even infeasible when a synthesis protocol for the material with the desired composition is not yet developed. In addition, the possible number of synthesizable zeolites is enormously large, with some of the structures found in the predicted crystallography open database (PCOD) possessing potentially much better characteristics. Selecting optimal candidate materials through predictive modeling is hence a very attractive proposition. Such screening studies have so far focused mostly on single-component adsorption of small, rigid, non-hydrogen-bonding molecules, such as H₂, CH₄, and CO₂. Screening sorbents and catalysts for complex mixtures composed of large, articulated molecules, where advanced algorithms are required for sampling the distribution of thousands of conformers, or polar, hydrogen-bonding molecules, where an accurate description of electrostatics and the resulting mixture non-idealities are of paramount importance, has so far been an intractable problem. Enabled by a multistep screening workflow, efficient sampling algorithms, accurate force fields, and a two-level parallel execution hierarchy utilizing up to 131,072 computer cores on a supercomputer, the inventors have engaged in high-throughput-screening for conversion of linear alkanes and slightly branched alkanes of more moderate molecular weight, e.g., having 18-30 carbon atoms.

The inventors have identified, through a large-scale, multi-step computational screening process, promising zeolite structures capable of providing a catalyst system that can provide adsorption that is up to two orders of magnitude better than current technology for linear and slightly branched alkanes with 18-30 carbon atoms, such as those encountered in petroleum refining. A suitable zeolite can possess a high affinity for linear alkanes but low affinity for branched isomers, so that the desired mono-branched products are not being cracked into smaller species.

The present disclosure describes a dewaxing process that comprises contacting, under hydroisomerization conditions, the hydrocarbon feedstock with a bi-functional catalyst system comprising one or more zeolites and a hydrogenation component. The framework types of the one or more zeolites can be selected to minimize cracking while maximize the hydroisomerization of linear alkanes. The hydrogenation component can comprise at least one Group VIII metal, Group VIB metal, or Group IB metal. At least a fraction of the waxy linear or slightly branched alkanes or side chains can be converted by contacting with the catalyst system.

Zeolites in the catalyst system can provide Brønsted acid functionality. The zeolites can be partially or fully proton-exchanged. The desired SiO₂/Al₂O₃ ratio can be very high, so that the acid strength is weak and cracking is minimized. The zeolites selected can contain a channel system of medium pore sizes that can be constructed from 10-12 member rings and characterized by a roughly symmetrical shape and a pore diameter of from about 0.45 nanometers (nm) to about 0.7 nm. For example, zeolites useful in the conversion process of the present disclosure can include one-dimensional structures of CAN, EUO, GON, and VET framework type zeolites or three-dimensional structures of WEN and ITH framework type zeolites. Zeolites of different framework types can also be used together, either as mixtures, stacked layers, separated, or any other configuration in which the feedstock is brought into contact with all of the zeolites simultaneously or sequentially. In order to improve catalyst utilization, the form of the zeolites can be made as small crystals or hierarchical materials with mesopores.

The hydrogenation component (e.g., Group VIII metals, Group VIB metals, or Group IB metals) can provide hydrogenation functionality. In an example, the hydrogenation component can be selected from Group VIII metals, such as from Pt, Pd, or mixtures thereof. The metals can be incorporated into a zeolite by any of the known techniques, such as occlusion, impregnation, and ion exchange, in either elemental form or as oxides, sulfides, or mixtures thereof. The amount of the hydrogenation component can be small, for example ranging from about 0.1 wt % to about 5 wt %, such as from about 0.2 wt % to about 1 wt % of the catalyst system.

The hydroisomerization process can be operated with added hydrogen, but can also be carried out in the absence of hydrogen. The reaction can occur at a temperature of about 473-773 K. The reaction can occur at a pressure of from about 0.1 MPa to about 20 MPa. The reaction can proceed at a liquid hourly space velocity (LHSV) of from about 0.1 hr⁻¹ to about 20 hr⁻¹. The lower end of these values of temperature, pressure, and LHSV can promote isomerization, while the higher end of these value ranges can promote cracking. The base oil product of the hydroisomerization reaction can further be hydrofinished and additized.

There is also provided herein a method for systematic discovery of zeolite framework types that are suitable for processes such as the dewaxing processes described above. The method of systematic discovery can comprise assembling a group of a plurality of candidate zeolite structures. Each candidate of the plurality of candidate zeolite structures can be evaluated based on several key quantities, and finally a composite score can be formed for each candidate. In one example, the plurality of candidate zeolite structures can include experimentally synthesized zeolites such as those deposited in the Inorganic Crystal Structure Database or the database maintained by IZA-SC. It further includes zeolite structures that have been predicted to be thermodynamically accessible but have not been synthesized yet, such as those deposited in the PCOD database and described in Pophale et al., Phys. Chem. Chem. Phys. 13, 12407 (2011), the entire disclosure of which is incorporated by reference as if reproduced herein in its entirety. The group of candidate structures can be first pruned using a fast procedure, such as geometric analysis or short calculations, or both, in order to eliminate structures that are unattractive for the target application. The surviving candidates can then be subjected to careful evaluation, at representative reaction conditions (for example for a prototypical multi-component feed mixture, at a temperature of 573 K and a pressure of 3 MPa), of a few quantities that are chosen to reflect different facets of the isomerization conversion: high affinity towards linear alkanes as indicated by the equilibrium loading of n-octadecane (Q_{nC18}), high adsorption selectivity of linear over branched alkanes as indicated by Equation [1],

$${S}_{B 3} = \frac{3 {Q}_{nC 18}}{{Q}_{2 mC 17} + {Q}_{4 mC 17} + {Q}_{22 d mC 16}} \qquad [ 1 ]$$

and low selectivity of linear alkanes of different molecular weights as indicated by Equation [2]

$${S}_{L 3} = \frac{\sqrt{{Q}_{nC 24} {Q}_{nC 30}}}{{Q}_{nC 18}} \qquad [ 2 ]$$

where Q_{2mC17}, Q_{4mC17}, Q_{22dmC16}, Q_{nC24}, and Q_{nC30} denote the equilibrium loadings of 2-methyl- and 4-methylheptadecane, 2,2-dimethylhexadecane, n-tetracosane, and n-triacontane, respectively. It has been found that the criteria of affinity toward linear alkanes and the adsorption selectivity of linear over branched alkanes have a larger effect on the performance of the zeolites, so in some examples a method of screening may be based only on those two criteria. However, the third criteria, selectivity of linear alkanes of different molecular weights, has been found to be beneficial in finding which known zeolite materials will be the best performers for a particular process.

The top performing zeolites can be selected to be those with largest values of a composite score according to Equation [3]:

P=Q_{nC18}×S_{B3}/S_{L3} [3]

The method of evaluating zeolite structures can rediscover previously known zeolites as being among those with the highest scores as well as identifies the zeolites described in the present disclosure.

### Example

The present disclosure can be further understood by reference to the following example which is offered by way of illustration. The present disclosure is not limited to the example given herein.

Mildly acidic zeolites with high separation factors for linear long-chain alkanes over their branched counterparts are very conducive to reducing cracking and increasing the fraction of branched species in the hydroisomerization process. Adsorption using the siliceous forms of the same zeolite framework types also can be desired in, for example, the process for separating branched alkanes used in transportation fuels from linear ones used to produce plasticizers and synthetic detergents, provided under the trade name MOLEX by UOP LLC of Des Plaines, Ill., USA. Previously, Dubbeldam et al. screened about 100 nanoporous materials for the adsorption of hexane and heptane isomers. In this Example, >330,000 zeolite structures in the IZA-SC and PCOD databases were screened. To represent the complex hydrocarbon feed, an equimolar mixture of n-octadecane (C18), n-tetracosane (C24), n-triacontane (C30), 2-methyl and 4-methylheptadecane (2C17 and 4C17), and 2,2-dimethylhexadecane (22C16) was employed. Three factors are considered to contribute to performance: (i) high affinity towards linear alkanes as indicated by Henry's constant, k_{H,C18}, at low pressure or loading, Q_{C18}, at P=3 MPa for C18; (ii) high selectivity for linear versus branched alkanes as indicated by S_{Bn}=3y_{C18}/(y_{2C17}+y_{22C16}) where n=0 or 3 and y=k_{H} or Q depending on state point; and (iii) low selectivity between linear alkanes of different lengths as indicated by S_{Ln}=(y_{C24}y_{c30})^{1/2}/y_{C18} so that a broad range of linear alkanes may be converted; the resulting performance metric is P_{HCn}=y_{C18}S_{Bn}/S_{Ln}.

The first screening step aims to reduce the number of candidate materials using relatively short simulations performed at T=573 K and the low-pressure limit. Screening the IZA-SC database reveals that six of the top-7 structures (ATO, MRE, MTT, AFO, MTW, and FER) are already known for isodewaxing. This strongly indicates that our screening procedure, although based solely on adsorption properties, is able to capture the essential characteristics responsible for high-performing hydroisomerization catalysts and can thus provide useful guidance for experimental synthesis of novel zeolite catalysts. Thus, the pool of candidate materials was expanded to include zeolite-like structures in the PCOD database. Materials that are among the top-64 in the IZA-SC database or top-1024 in the PCOD database in any of the three performance indicators (for a total of 103 and 2835 structures, respectively) are retained for longer simulations in the low-pressure limit and for an equimolar mixture of all six components at T=573 K and p=3 MPa, a typical operation condition for the hydroisomerization conversion.

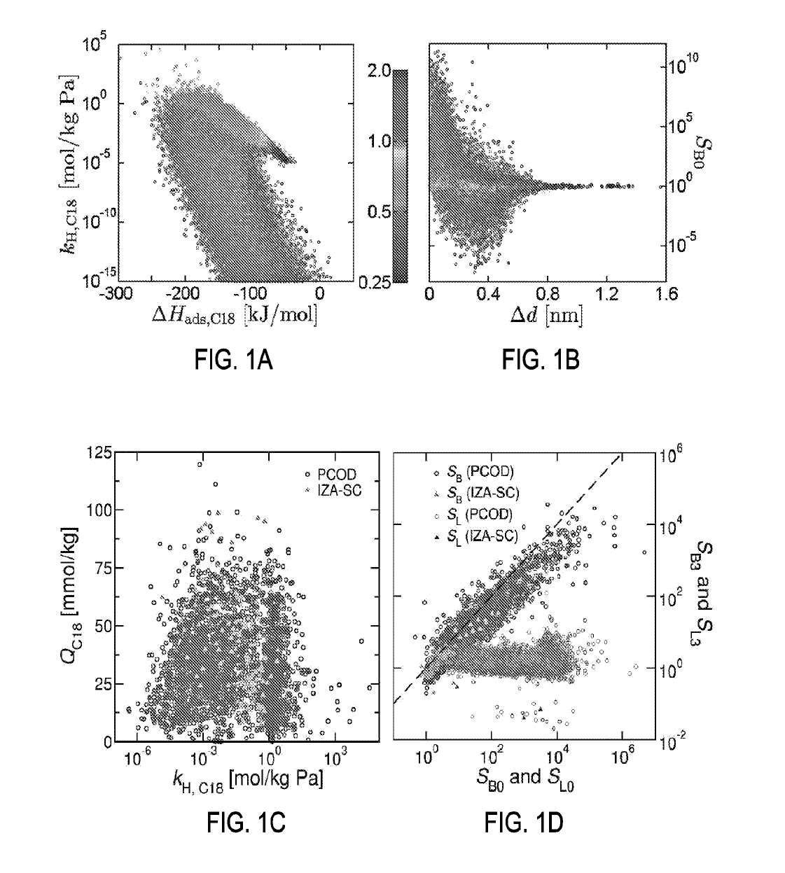

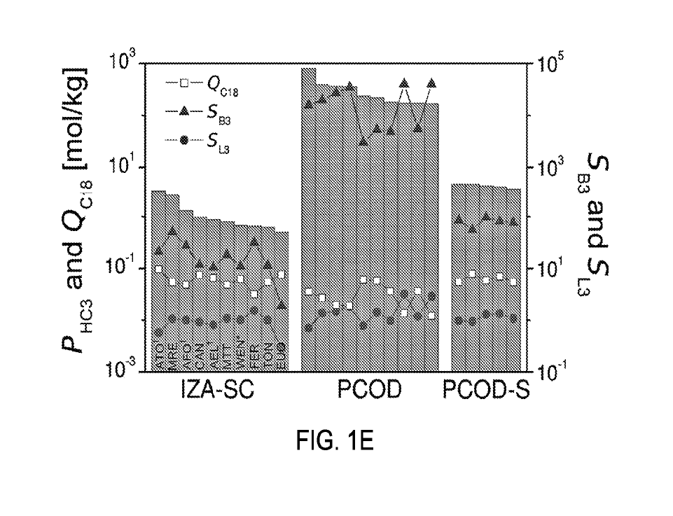

FIG. 1a illustrates the relationships between k_{H} and the adsorption enthalpy, ΔH_{ads}, for C18 adsorption in the low-pressure limit. A more favorable ΔH_{ads} generally leads to a larger k_{H}, but the correlation is fairly weak. This reflects enthalpy-entropy compensation where a smaller entropic loss in a wider channel can compensate for a smaller enthalpic gain, as seen by data points for frameworks with larger free channel diameters being more frequently located toward the upper right corner in FIG. 1a. A strong affinity for C18, as indicated by large k_{H,C18}, is well correlated with an even stronger affinity for C24 and C30, i.e., large S_{L0}, but not correlated with a preference for linear versus branched alkanes, e.g., large S_{B0}. It was found that S_{B0} is best correlated with the pore bumpiness, Δd=d_{incl}−d_{free} where the first and second terms are the diameter of the largest sphere that can be placed anywhere along a given channel, and the diameter of the largest sphere that can roam freely through the channel. As shown in FIG. 1b, a narrow and smooth channel yields high selectivity for linear alkanes, whereas a narrow but bumpy channel with Δd≈0.3 nm leads to inverse selectivity where adsorption of the branched alkanes is preferred.

FIGS. 1c and 1d compares adsorption characteristics calculated in the infinite dilution limit and at hydroisomerization conditions. For both IZA-SC and PCOD structures, there is only a weak correlation between the low-pressure affinity and the high-pressure loading because larger pores allow for high loading but are not most favorable for individual sorbates (see FIG. 1a). The two selectivities exhibit markedly different trends as pressure is increased: S_{B} remains nearly constant, whereas S_{L} decreases significantly for the majority of zeolites. Longer alkanes have much smaller vapor pressures and, correspondingly, the free energy penalty for them to leave the liquid phase is larger than for shorter alkanes and compensates for the more favorable free energy of adsorption. As a result, S_{L} reduces to around 1-10 at p=3 MPa. The complex pressure dependency of S_{L} illustrates the shortcomings of using infinite-dilution data to extrapolate material performance at other conditions.

FIG. 1e compares the top-10 structures (as ranked by P_{HC3}) from each database. Numerical values are provided in Table 1.

TABLE 1
Numerical data for hydrocarbon adsorption (k_{H, C18} in mol/kg Pa), Q_{C18} in mmol/kg) for
the top-10 IZA-SC and PCOD structures based on simulations in the low-pressure limit and at p =
3 MPa. The last 5 rows show the PCOD structures with the highest R₃ that have similar selectivities
to those of the top-10 IZA-SC zeolites (S_{B3} < 100 and S_{L3} > 0.9).

| | low-pressure limit | | | | p = 3 MPa | | | |
|---|---|---|---|---|---|---|---|---|
| Name | k_{H, C18} | S_{B0} | S_{L0} | R₀ | Q_{C18} | S_{B3} | S_{L3} | R₃ |
| ATO^{†} | 6.2 × 10⁻¹ | 65 | 6400 | 2 | 95 | 21 | 0.58 | 1 |
| MRE | 3.6 × 10⁰ | 23 | 14000 | 3 | 54 | 51 | 1.1 | 2 |
| AFO^{†} | 1.4 × 10⁻⁴ | 330 | 21 | 5 | 48 | 27 | 1.0 | 3 |
| CAN | 1.4 × 10⁰ | 45 | 7100 | 1 | 75 | 12 | 0.91 | 4 |
| AEL^{†} | 4.5 × 10 ⁻⁴ | 7.9 | 280 | 94 | 65 | 11 | 0.81 | 5 |
| MTT | 7.1 × 10⁻³ | 70 | 110 | 4 | 48 | 18 | 1.1 | 6 |
| WEN* | 1.4 × 10⁻⁵ | 27 | 1.8 | 47 | 62 | 11 | 1.0 | 7 |
| FER | 9.5 × 10⁻⁴ | 110 | 88 | 7 | 32 | 32 | 1.5 | 8 |
| TON | 4.4 × 10⁻³ | 8.0 | 79 | 19 | 55 | 11 | 1.0 | 9 |
| EUO | 4.3 × 10⁻³ | 1.4 | 8.6 | 12 | 76 | 1.9 | 0.29 | 10 |
| MTW | 2.5 × 10⁰ | 5.5 | 9000 | 6 | 40 | 2.8 | 1.4 | 28 |
| GON | 2.3 × 10⁰ | 4.6 | 10000 | 8 | 35 | 2.7 | 1.3 | 34 |
| VET | 1.2 × 10⁰ | 4.2 | 5400 | 9 | 28 | 2.1 | 1.5 | 47 |
| ITH | 5.2 × 10⁻³ | 5.5 | 34 | 10 | 40 | 1.4 | 1.5 | 52 |
| 8113534 | 2.4 × 10⁻³ | 6.1 × 10⁵ | 5.0 × 10² | 18 | 36 | 16000 | 0.71 | 1 |
| 8296636 | 3.7 × 10⁻² | 1.4 × 10⁴ | 3.9 × 10² | 31 | 27 | 20000 | 1.4 | 2 |
| 8302206 | 9.9 × 10⁻⁴ | 1.4 × 10⁵ | 1.2 × 10² | 35 | 20 | 27000 | 1.5 | 3 |
| 8319806 | 1.4 × 10⁻⁵ | 5.3 × 10³ | 2.0 × 10¹ | 734 | 19 | 35000 | 1.8 | 4 |
| 8165762 | 4.6 × 10⁻⁴ | 2.5 × 10⁴ | 5.8 × 10¹ | 104 | 61 | 3100 | 0.79 | 5 |
| 8302179 | 4.0 × 10⁻⁴ | 4.1 × 10⁴ | 3.1 × 10¹ | 66 | 58 | 5300 | 1.4 | 6 |
| 8121102 | 4.4 × 10⁻⁴ | 3.3 × 10⁴ | 2.3 × 10² | 187 | 36 | 4900 | 0.98 | 7 |
| 8276859 | 7.6 × 10⁻⁵ | 2.2 × 10⁵ | 6.0 × 10¹ | 91 | 14 | 40000 | 3.2 | 8 |
| 8149581 | 5.1 × 10⁻⁴ | 1.6 × 10⁴ | 2.1 × 10¹ | 79 | 36 | 5600 | 1.2 | 9 |
| 8244356 | 6.5 × 10⁻⁵ | 4.2 × 10⁴ | 4.2 × 10⁰ | 50 | 12 | 40000 | 2.9 | 10 |
| 8246562 | 5.4 × 10⁻³ | 1.9 × 10⁶ | 1.9 × 10² | 9 | 8.1 | 40000 | 4.6 | 25 |
| 8325576 | 6.9 × 10³ | 6.0 × 10⁵ | 2.3 × 10⁵ | 3 | 14 | 2400 | 2.8 | 128 |
| 8280370 | 4.4 × 10⁻³ | 4.7 × 10⁵ | 1.6 × 10¹ | 8 | 2.2 | 40000 | 10 | 171 |
| 8276346 | 9.8 × 10⁻¹ | 9.6 × 10³ | 2.4 × 10² | 10 | 13 | 830 | 2.1 | 267 |
| 8161406 | 1.5 × 10⁻² | 4.6 × 10⁴ | 3.0 × 10⁰ | 5 | 3.2 | 2700 | 4.5 | 520 |
| 8325781 | 7.6 × 10⁰ | 3.7 × 10⁴ | 1.3 × 10³ | 6 | 6.2 | 600 | 2.7 | 630 |
| 8182003 | 2.8 × 10⁰ | 4.7 × 10⁶ | 7.8 × 10³ | 4 | 1.9 | 1600 | 7.2 | 1113 |
| 8328013 | 1.5 × 10⁰ | 1.0 × 10⁹ | 9.5 × 10² | 2 | 0.47 | 40000 | 67 | 1301 |
| 8316501 | 9.3 × 10⁰ | 7.6 × 10³ | 3.5 × 10² | 7 | 49 | 1.0 | 1.2 | 2200 |
| 8295863 | 1.3 × 10⁰ | 5.9 × 10⁹ | 4.8 × 10³ | 1 | 0.058 | 40000 | 120 | 2410 |
| 8083868 | 1.1 × 10⁻⁵ | 19 | 2.1 | 2241 | 55 | 85 | 0.98 | 276 |
| 8216857 | 9.9 × 10⁻¹ | 83 | 8800 | 474 | 78 | 56 | 0.94 | 278 |
| 8165707 | 8.1 × 10⁻⁵ | 12⁰ | 5.7 | 1005 | 59 | 98 | 1.3 | 290 |
| 8285996 | 6.6 × 10⁻⁵ | 39 | 32 | 2304 | 69 | 82 | 1.3 | 298 |
| 8133653 | 1.1 × 10⁻² | 36⁰ | 170 | 311 | 54 | 77 | 1.1 | 313 |

*idealized siliceous structure.
^{†}Aluminophosphate.

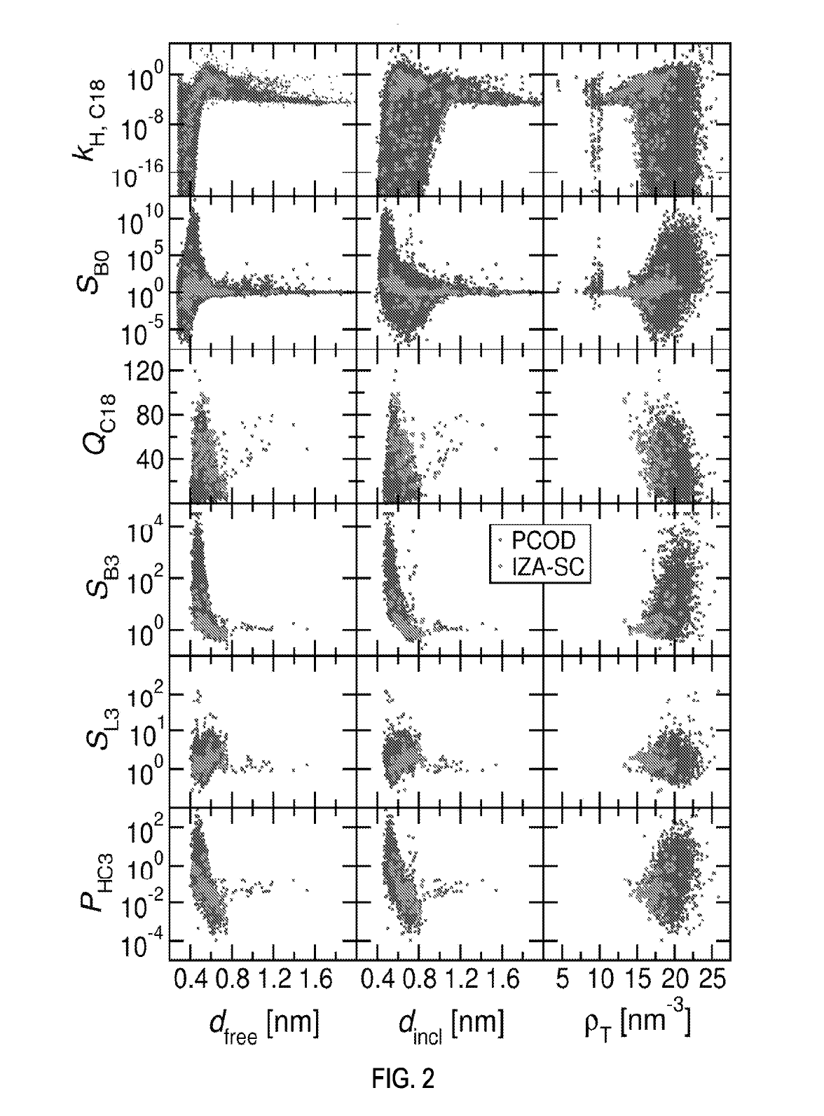

The top 3 (ATO, MRE, and AFO), and another four from the top 10 (AEL, MTT, FER, and TON) IZA-SC structures are known to excel for this petrochemical application; fourth and seventh ranked CAN and WEN* have performance indicators resembling the other structures, whereas EUO yields a lower S_{B3} that is compensated by a more favorable S_{L3}. Top-ranked ATO features the highest Q_{C18}, the second lowest (favorable) S_{L3}, and the fourth highest S_{B3}. The largest variation among the top-10 IZA-SC structures is found for S_{B3} that range from 2-50, whereas Q_{C18} and S_{L3} vary only by factors of 3 and 5. The top-10 PCOD structures exhibit P_{HC3} values that are about two orders of magnitude higher than those for the high-performing IZA-SC structures. This dramatically improved performance arises mainly from their exceptionally high S_{B3} values, whereas their Q_{C18} and S_{L3} values tend to be slightly less favorable than for the IZA-SC structures (as indicated by the five structures labeled PCOD-S, there are also structures that match the Q_{C18} and S_{L3} values of the top IZA-SC performers and still outperform them). The performance differences are much larger in the low-pressure limit where some PCOD structures yield k_{H,C18} and S_{B0} values that exceed those of the IZA-SC structures by 3 and 7 orders of magnitude, respectively (see Table 1 and FIG. 2).

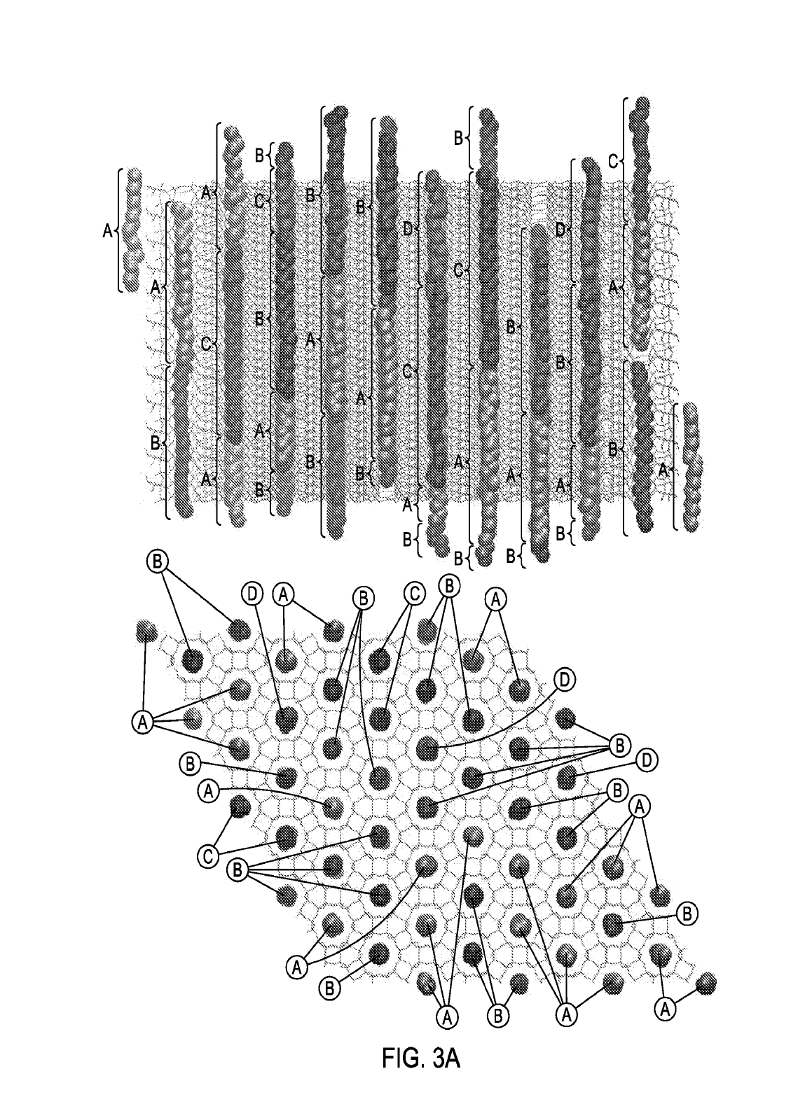

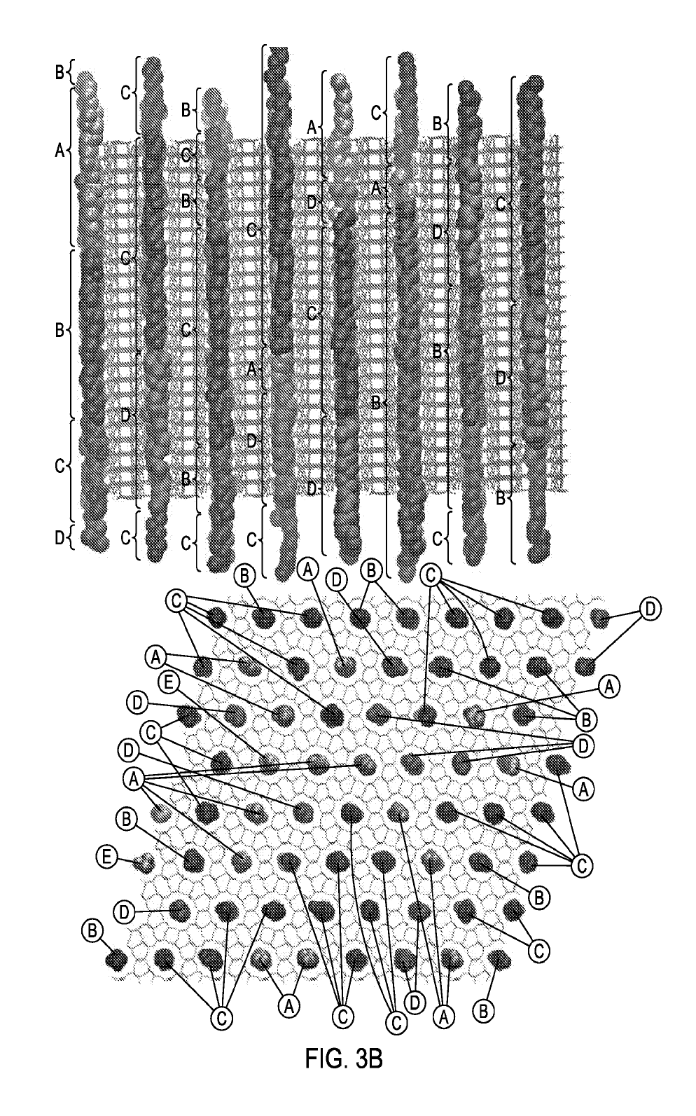

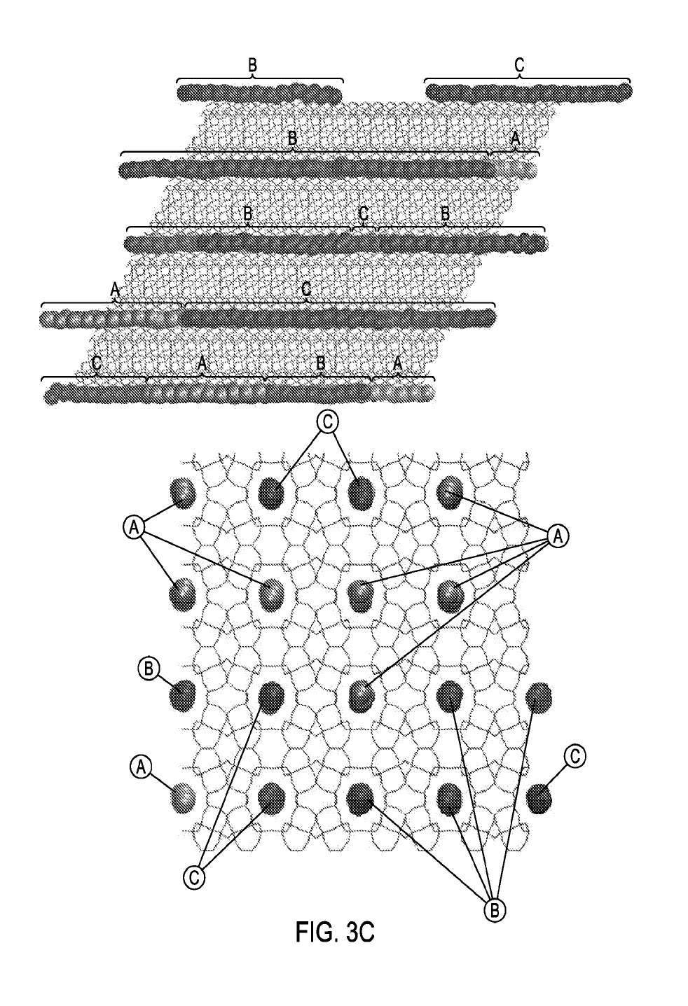

In FIG. 3, the arrangement of the adsorbed hydrocarbons in ATO, MTW, and PCOD-8113534 are compared. As can be seen, the top-performing structures all possess one-dimensional 12-ring channel architectures and these systems are near saturation loading at p=3 MPa. ATO and PCOD-8113534 are the top-ranked IZA-SC and PCOD structure and also perform exceptionally well in the low-pressure limit. MTW is a framework type and is the sixth-ranked structure at low pressure, but only ranks 28th at p=3 MPa because of a large decrease in S_{B}. The S_{B3} values for these three structures are 21, 2.8, and 16000, respectively. As expected from the large S_{B3}, in PCOD-8113534's elliptic channels with a semi-minor axis length of only 0.47 nm the linear alkanes are highly confined with a preference to have their zig-zag plane aligned along the semi-major elliptic axis and exhibit very few gauched effects at the chain termini. ATO's channels possess a cross section resembling a regular hexagon with d_{free}=0.57 nm, and the zig-zag plane of the alkanes is free to rotate; in addition to end-gauche defects, kink defects (gtg′) are also observed. The even larger elliptic channels in MTW afford sufficient room for few 22C16 molecules and gttg′ kinks in linear chains.

### Methods

Framework Structures.

The IZA-SC database used in the screening comprises a set of idealized framework structures and other experimentally determined structures. The database is provided by Baerlocher & McCusker at http://www.iza-structure.org/databases as “Database of zeolite structures,” the entire content of which is incorporated by reference as if reproduced herein in their entirety. The idealized structure for each framework type is obtained by geometric refinement with prescribed interatomic distances, assuming a (hypothetical) SiO₂ composition, and in the highest possible symmetry space group of the framework type. The experimental structures are included if they contain only O, Si, Al, P, or H atoms. Solvent molecules and ions were removed, and partial occupation were randomly assigned at the unit cell level. A larger PCOD database, provided by Pophale, R., Cheeseman, P. A. & Deem, M. W. “A database of new zeolite-like materials,”. Phys. Chem. Chem. Phys. 13, 12407-12412 (2011), incorporated by reference as if reproduced herein in its entirety, was constructed by enumerating space groups, unit cells, density and sampling coordinates of Si atoms in the irreducible unit. The resulting 2.6 million candidate structures were geometry optimized and, based on an energetic criterion, 331,172 structures are considered as thermodynamically accessible. A performance rank is only given to structures that have accessible pores/channels.

Simulation Methods.

The transferable potentials for phase equilibria (TraPPE) force field are utilized to describe the sorbate-sorbate and sorbate-zeolite interactions modelled via Lennard-Jones (Li) and Coulomb potentials. In our simulations, the zeolite frameworks are assumed to be rigid, while sorbate molecules sample angle bending and dihedral motions. To improve computational efficiency, grid files for the interaction energy of a test particle with the zeolite were generated in a manner that contains the repulsive LJ, attractive LJ, and the short- and long-range Coulomb components, but is independent of the specific force field parameters for the sorbate molecule; therefore the same grid files can facilitate screening calculations for other applications. Configurational-bias Monte Carlo simulations in the grand-canonical ensemble (CB-GCMC) were used to compute the sorbate loadings as function of the partial pressure for the alkanes using chemical potentials determined from liquid-phase simulations in the isobaric-isothermal ensemble. In the infinite-dilution limit, these CB-GCMC simulations also yield directly k_{H} and ΔH_{ads}. To carry out the energy grid tabulation and CB-GCMC simulations in a high-throughput fashion, a two-level parallel execution hierarchy exploring simultaneously 2⁷ to 2¹⁴ zeolite structures and accelerating the simulations for each structure by spreading the computational load over 2² to 2⁸ computer cores was employed. These massively-parallel screening calculations can be performed on a supercomputer.

### Exemplary Embodiments

To better illustrate the processes and zeolite materials of the present disclosure, a non-limiting list of EMBODIMENTS is provided here:

EMBODIMENT 1 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one or more zeolites, the catalyst system comprising one or more zeolites with one-dimensional channel systems selected from the group consisting of a CAN-type zeolite, a EUO-type zeolite, a GON-type zeolite, and a VET-type zeolite.

EMBODIMENT 2 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one or more zeolites, the catalyst system comprising one or more zeolites with three-dimensional channel systems selected from the group consisting of a WEN-type zeolite and an ITH-type zeolite.

EMBODIMENT 3 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one or more zeolites, the catalyst system comprising one or more zeolites with one-dimensional channel systems including at least one zeolite selected from the PCOD database having a PCOD identifier selected from the group consisting of 8113534, 8296636, 8302206, 8165762, 8302179, 8121102, 8276859, 8149581, 8244356, 8296363, 8321948, 8138688, 8223156, 8245547, 8223914, 8305046, 8152848, 8234126, 8190755, 8127992, 8152156, 8305042, 8137831, 8246562, 8296721, 8258975, 8326325, 8247653, 8165791, 8118469, 8186880, 8203701, 8216070, 8324735, 8318543, 8324823, 8296863, 8304854, 8322003, 8295642, 8205808, 8164336, 8129506, 8322048, 8119337, 8255575, 8159638, 8269679, 8153524, 8319059, 8026125, 8273431, 8295955, 8269459, 8170433, 8164121, 8326255, 8281376, 8301873, 8165656, 8277094, 8233560, 8137616, 8149587, 8169792, 8293991, 8262350, 8264198, 8115891, 8262902, 8306970, 8226024, 8322924, 8152100, 8277433, 8218629, 8054782, 8120251, 8163400, 8247952, 8109723, 8234406, 8216438, 8296333, 8216552, 8150035, 8259735, 8130003, 8137562, 8279697, 8163965, 8324742, 8155533, 8148176, 8072540, 8217078, 8297845, 8165033, 8276974, 8298516, 8054597, 8082795, 8203181, 8326100, 8139317, 8165538, 8326372, 8254444, 8167956, 8054214, 8193268, 8304867, 8223456, 8045653, 8302209, 8219004, 8306237, 8272811, 8157648, 8326099, 8166074, 8158356, 8325576, 8158769, 8121081, 8324818, 8323141, 8247317, 8166112, 8073568, 8277341, 8222738, 8141396, 8232287, 8245510, 8254068, 8152150, 8148869, 8204023, 8326245, 8306845, 8219078, 8164071, 8306066, 8111747, 8204010, 8161093, 8263690, 8205511, 8097894, 8059282, 8329494, 8219685, 8164344, 8311634, 8238847, 8165831, 8134613, 8219214, 8030963, 8141091, 8322611, 8169582, 8282106, 8326097, 8222264, 8030519, 8005350, 8254017, 8114704, 8183709, 8137450, 8274361, 8233586, 8119910, 8264279, 8323852, 8243329, 8254550, 8291455, 8219891, 8308806, 8326257, 8325769, 8004705, 8321111, 8121189, 8147634, 8166054, 8301299, 8279593, 8123558, 8183228, 8281874, 8204430, 8054794, 8124909, 8323165, 8294533, 8148507, 8008796, 8128538, 8233864, 8165171, 8262781, 8231696, 8304734, 8263651, 8076700, 8226706, 8257181, 8222180, 8136633, 8247289, 8228296, 8074110, 8160689, 8245586, 8301408, 8257704, 8281264, 8282839, 8293427, 8246429, 8135370, 8124669, 8152114, 8276300, 8318439, 8304970, 8114386, 8233041, 8311771, 8227536, 8215446, 8271288, 8234171, 8175996, 8311227, 8323124, 8201592, 8159102, 8137417, 8151237, 8160333, 8165896, 8151013, 8289645, 8247005, 8029604, 8222883, 8131207, 8095377, 8175192, 8276346, 8205710, 8279368, 8175541, 8222024, 8326153, 8319790, 8310983, 8122614, 8083868, 8119832, 8216857, 8145217, 8292039, 8221747, 8325766, 8320603, 8323818, 8076935, 8152101, 8277551, 8049792, 8274130, 8165707, 8141266, 8277398, 8183844, 8304796, 8063993, 8046781, 8285996, 8313513, and 8053941.

EMBODIMENT 4 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one or more zeolites, the catalyst system comprising one or more zeolites with two-dimensional channel systems selected from the PCOD database having a PCOD identifier selected from the group consisting of 8178377, 8285531, 8280370, 8285533, and 8274377.

EMBODIMENT 5 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one or more zeolites, the catalyst system comprising one or more zeolites with three-dimensional channel systems selected from the PCOD database having a PCOD identifier selected from the group consisting of 8319806, 8311374, 8316340, 8305889, 8065252, 8305207, 8308011, and 8308120.

EMBODIMENT 6 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one of: (a) one or more zeolites with one-dimensional channel systems selected from the group consisting of a CAN-type zeolite, an EUO-type zeolite, a GON-type zeolite, and a VET-type zeolite; or (b) one or more zeolites with three-dimensional channel systems selected from the group consisting of a WEN-type zeolite and an ITH-type zeolite.

EMBODIMENT 7 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one or more zeolites, the catalyst system comprising one of: (a) one or more zeolites with one-dimensional channel systems selected from the PCOD database having a PCOD identifier selected from the group consisting of 8113534, 8296636, 8302206, 8165762, 8302179, 8121102, 8276859, 8149581, 8244356, 8296363, 8321948, 8138688, 8223156, 8245547, 8223914, 8305046, 8152848, 8234126, 8190755, 8127992, 8152156, 8305042, 8137831, 8246562, 8296721, 8258975, 8326325, 8247653, 8165791, 8118469, 8186880, 8203701, 8216070, 8324735, 8318543, 8324823, 8296863, 8304854, 8322003, 8295642, 8205808, 8164336, 8129506, 8322048, 8119337, 8255575, 8159638, 8269679, 8153524, 8319059, 8026125, 8273431, 8295955, 8269459, 8170433, 8164121, 8326255, 8281376, 8301873, 8165656, 8277094, 8233560, 8137616, 8149587, 8169792, 8293991, 8262350, 8264198, 8115891, 8262902, 8306970, 8226024, 8322924, 8152100, 8277433, 8218629, 8054782, 8120251, 8163400, 8247952, 8109723, 8234406, 8216438, 8296333, 8216552, 8150035, 8259735, 8130003, 8137562, 8279697, 8163965, 8324742, 8155533, 8148176, 8072540, 8217078, 8297845, 8165033, 8276974, 8298516, 8054597, 8082795, 8203181, 8326100, 8139317, 8165538, 8326372, 8254444, 8167956, 8054214, 8193268, 8304867, 8223456, 8045653, 8302209, 8219004, 8306237, 8272811, 8157648, 8326099, 8166074, 8158356, 8325576, 8158769, 8121081, 8324818, 8323141, 8247317, 8166112, 8073568, 8277341, 8222738, 8141396, 8232287, 8245510, 8254068, 8152150, 8148869, 8204023, 8326245, 8306845, 8219078, 8164071, 8306066, 8111747, 8204010, 8161093, 8263690, 8205511, 8097894, 8059282, 8329494, 8219685, 8164344, 8311634, 8238847, 8165831, 8134613, 8219214, 8030963, 8141091, 8322611, 8169582, 8282106, 8326097, 8222264, 8030519, 8005350, 8254017, 8114704, 8183709, 8137450, 8274361, 8233586, 8119910, 8264279, 8323852, 8243329, 8254550, 8291455, 8219891, 8308806, 8326257, 8325769, 8004705, 8321111, 8121189, 8147634, 8166054, 8301299, 8279593, 8123558, 8183228, 8281874, 8204430, 8054794, 8124909, 8323165, 8294533, 8148507, 8008796, 8128538, 8233864, 8165171, 8262781, 8231696, 8304734, 8263651, 8076700, 8226706, 8257181, 8222180, 8136633, 8247289, 8228296, 8074110, 8160689, 8245586, 8301408, 8257704, 8281264, 8282839, 8293427, 8246429, 8135370, 8124669, 8152114, 8276300, 8318439, 8304970, 8114386, 8233041, 8311771, 8227536, 8215446, 8271288, 8234171, 8175996, 8311227, 8323124, 8201592, 8159102, 8137417, 8151237, 8160333, 8165896, 8151013, 8289645, 8247005, 8029604, 8222883, 8131207, 8095377, 8175192, 8276346, 8205710, 8279368, 8175541, 8222024, 8326153, 8319790, 8310983, 8122614, 8083868, 8119832, 8216857, 8145217, 8292039, 8221747, 8325766, 8320603, 8323818, 8076935, 8152101, 8277551, 8049792, 8274130, 8165707, 8141266, 8277398, 8183844, 8304796, 8063993, 8046781, 8285996, 8313513, and 8053941; (b) one or more zeolites with two-dimensional channel systems selected from the PCOD database having a PCOD identifier selected from the group consisting of 8178377, 8285531, 8280370, 8285533, and 8274377; or (c) one or more zeolites with three-dimensional channel systems selected from the PCOD database having a PCOD identifier selected from the group consisting of 8319806, 8311374, 8316340, 8305889, 8065252, 8305207, 8308011, and 8308120.

EMBODIMENT 8 can include subject matter (such as an apparatus, a device, a method, or one or more means for performing acts), such as can include a process for conversion of at least a fraction of one or more linear or slightly branched alkanes of a hydrocarbon feedstock. The subject matter can include contacting the hydrocarbon feedstock with a catalyst system comprising one or more zeolites, the catalyst system comprising one of: (a) one or more zeolites with one-dimensional channel systems selected from the group consisting of a CAN-type zeolite, an EUO-type zeolite, a GON-type zeolite, and a VET-type zeolite; (b) one or more zeolites with three-dimensional channel systems selected from the group consisting of a WEN-type zeolite and an ITH-type zeolite; (c) one or more zeolites with one-dimensional channel systems selected from the PCOD database having a PCOD identifier selected from the group consisting of 8113534, 8296636, 8302206, 8165762, 8302179, 8121102, 8276859, 8149581, 8244356, 8296363, 8321948, 8138688, 8223156, 8245547, 8223914, 8305046, 8152848, 8234126, 8190755, 8127992, 8152156, 8305042, 8137831, 8246562, 8296721, 8258975, 8326325, 8247653, 8165791, 8118469, 8186880, 8203701, 8216070, 8324735, 8318543, 8324823, 8296863, 8304854, 8322003, 8295642, 8205808, 8164336, 8129506, 8322048, 8119337, 8255575, 8159638, 8269679, 8153524, 8319059, 8026125, 8273431, 8295955, 8269459, 8170433, 8164121, 8326255, 8281376, 8301873, 8165656, 8277094, 8233560, 8137616, 8149587, 8169792, 8293991, 8262350, 8264198, 8115891, 8262902, 8306970, 8226024, 8322924, 8152100, 8277433, 8218629, 8054782, 8120251, 8163400, 8247952, 8109723, 8234406, 8216438, 8296333, 8216552, 8150035, 8259735, 8130003, 8137562, 8279697, 8163965, 8324742, 8155533, 8148176, 8072540, 8217078, 8297845, 8165033, 8276974, 8298516, 8054597, 8082795, 8203181, 8326100, 8139317, 8165538, 8326372, 8254444, 8167956, 8054214, 8193268, 8304867, 8223456, 8045653, 8302209, 8219004, 8306237, 8272811, 8157648, 8326099, 8166074, 8158356, 8325576, 8158769, 8121081, 8324818, 8323141, 8247317, 8166112, 8073568, 8277341, 8222738, 8141396, 8232287, 8245510, 8254068, 8152150, 8148869, 8204023, 8326245, 8306845, 8219078, 8164071, 8306066, 8111747, 8204010, 8161093, 8263690, 8205511, 8097894, 8059282, 8329494, 8219685, 8164344, 8311634, 8238847, 8165831, 8134613, 8219214, 8030963, 8141091, 8322611, 8169582, 8282106, 8326097, 8222264, 8030519, 8005350, 8254017, 8114704, 8183709, 8137450, 8274361, 8233586, 8119910, 8264279, 8323852, 8243329, 8254550, 8291455, 8219891, 8308806, 8326257, 8325769, 8004705, 8321111, 8121189, 8147634, 8166054, 8301299, 8279593, 8123558, 8183228, 8281874, 8204430, 8054794, 8124909, 8323165, 8294533, 8148507, 8008796, 8128538, 8233864, 8165171, 8262781, 8231696, 8304734, 8263651, 8076700, 8226706, 8257181, 8222180, 8136633, 8247289, 8228296, 8074110, 8160689, 8245586, 8301408, 8257704, 8281264, 8282839, 8293427, 8246429, 8135370, 8124669, 8152114, 8276300, 8318439, 8304970, 8114386, 8233041, 8311771, 8227536, 8215446, 8271288, 8234171, 8175996, 8311227, 8323124, 8201592, 8159102, 8137417, 8151237, 8160333, 8165896, 8151013, 8289645, 8247005, 8029604, 8222883, 8131207, 8095377, 8175192, 8276346, 8205710, 8279368, 8175541, 8222024, 8326153, 8319790, 8310983, 8122614, 8083868, 8119832, 8216857, 8145217, 8292039, 8221747, 8325766, 8320603, 8323818, 8076935, 8152101, 8277551, 8049792, 8274130, 8165707, 8141266, 8277398, 8183844, 8304796, 8063993, 8046781, 8285996, 8313513, and 8053941; (d) one or more zeolites with two-dimensional channel systems selected from the PCOD database having a PCOD identifier selected from the group consisting of 8178377, 8285531, 8280370, 8285533, and 8274377; or (e) one or more zeolites with three-dimensional channel systems selected from the PCOD database having a PCOD identifier selected from the group consisting of 8319806, 8311374, 8316340, 8305889, 8065252, 8305207, 8308011, and 8308120.

EMBODIMENT 9 can include, or can optionally be combined with, the subject matter of one or any combination of EMBODIMENTS 1-8, to optionally include the channel systems of the zeolite having pore diameters ranging from about 0.45 nanometers to about 0.7 nanometers.

EMBODIMENT 10 can include, or can optionally be combined with, the subject matter of one or any combination of EMBODIMENTS 1-9, to optionally include the catalyst system further comprising a hydrogenation component.

EMBODIMENT 11 can include, or can optionally be combined with, the subject matter of one or any combination of EMBODIMENTS 1-10, to optionally include the hydrogenation component comprising at least one of: one or more Group VIII metals, one or more Group VIB metals, and one or more Group IB metals.

EMBODIMENT 12 can include, or can optionally be combined with, the subject matter of one or any combination of EMBODIMENTS 1-11, to optionally include the Group VIII metals comprising platinum, palladium, or mixtures thereof.

EMBODIMENT 13 can include, or can optionally be combined with, the subject matter of one or any combination of EMBODIMENTS 1-12, to optionally include the contacting being performed at hydroisomerization conditions with added hydrogen.

EMBODIMENT 14 can include, or can optionally be combined with, the subject matter of one or any combination of EMBODIMENTS 1-13, to optionally include the contacting being performed at a temperature of 473-773 K.

EMBODIMENT 15 can include, or can optionally be combined with, the subject matter of one or any combination of EMBODIMENTS 1-14, to optionally include the contacting being performed at a pressure of 0.1-20 MPa.

EMBODIMENT 16 can include, or can optionally be combined with, the subject matter of one or any combination of EMBODIMENTS 1-15, to optionally include the contacting being performed at a liquid hourly space velocity of 0.1-20 hr⁻¹.

EMBODIMENT 17 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one or more zeolites with one-dimensional channel systems selected from the group consisting of a CAN-type zeolite, a EUO-type zeolite, a GON-type zeolite, and a VET-type zeolite.

EMBODIMENT 18 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one or more zeolites with three-dimensional channel systems selected from the group consisting of a WEN-type zeolite and an ITH-type zeolite.

EMBODIMENT 19 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one or more zeolites with one-dimensional channel systems including at least one zeolite selected from the PCOD database having a PCOD identifier selected from the group consisting of 8113534, 8296636, 8302206, 8165762, 8302179, 8121102, 8276859, 8149581, 8244356, 8296363, 8321948, 8138688, 8223156, 8245547, 8223914, 8305046, 8152848, 8234126, 8190755, 8127992, 8152156, 8305042, 8137831, 8246562, 8296721, 8258975, 8326325, 8247653, 8165791, 8118469, 8186880, 8203701, 8216070, 8324735, 8318543, 8324823, 8296863, 8304854, 8322003, 8295642, 8205808, 8164336, 8129506, 8322048, 8119337, 8255575, 8159638, 8269679, 8153524, 8319059, 8026125, 8273431, 8295955, 8269459, 8170433, 8164121, 8326255, 8281376, 8301873, 8165656, 8277094, 8233560, 8137616, 8149587, 8169792, 8293991, 8262350, 8264198, 8115891, 8262902, 8306970, 8226024, 8322924, 8152100, 8277433, 8218629, 8054782, 8120251, 8163400, 8247952, 8109723, 8234406, 8216438, 8296333, 8216552, 8150035, 8259735, 8130003, 8137562, 8279697, 8163965, 8324742, 8155533, 8148176, 8072540, 8217078, 8297845, 8165033, 8276974, 8298516, 8054597, 8082795, 8203181, 8326100, 8139317, 8165538, 8326372, 8254444, 8167956, 8054214, 8193268, 8304867, 8223456, 8045653, 8302209, 8219004, 8306237, 8272811, 8157648, 8326099, 8166074, 8158356, 8325576, 8158769, 8121081, 8324818, 8323141, 8247317, 8166112, 8073568, 8277341, 8222738, 8141396, 8232287, 8245510, 8254068, 8152150, 8148869, 8204023, 8326245, 8306845, 8219078, 8164071, 8306066, 8111747, 8204010, 8161093, 8263690, 8205511, 8097894, 8059282, 8329494, 8219685, 8164344, 8311634, 8238847, 8165831, 8134613, 8219214, 8030963, 8141091, 8322611, 8169582, 8282106, 8326097, 8222264, 8030519, 8005350, 8254017, 8114704, 8183709, 8137450, 8274361, 8233586, 8119910, 8264279, 8323852, 8243329, 8254550, 8291455, 8219891, 8308806, 8326257, 8325769, 8004705, 8321111, 8121189, 8147634, 8166054, 8301299, 8279593, 8123558, 8183228, 8281874, 8204430, 8054794, 8124909, 8323165, 8294533, 8148507, 8008796, 8128538, 8233864, 8165171, 8262781, 8231696, 8304734, 8263651, 8076700, 8226706, 8257181, 8222180, 8136633, 8247289, 8228296, 8074110, 8160689, 8245586, 8301408, 8257704, 8281264, 8282839, 8293427, 8246429, 8135370, 8124669, 8152114, 8276300, 8318439, 8304970, 8114386, 8233041, 8311771, 8227536, 8215446, 8271288, 8234171, 8175996, 8311227, 8323124, 8201592, 8159102, 8137417, 8151237, 8160333, 8165896, 8151013, 8289645, 8247005, 8029604, 8222883, 8131207, 8095377, 8175192, 8276346, 8205710, 8279368, 8175541, 8222024, 8326153, 8319790, 8310983, 8122614, 8083868, 8119832, 8216857, 8145217, 8292039, 8221747, 8325766, 8320603, 8323818, 8076935, 8152101, 8277551, 8049792, 8274130, 8165707, 8141266, 8277398, 8183844, 8304796, 8063993, 8046781, 8285996, 8313513, and 8053941.

EMBODIMENT 20 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one or more zeolites with two-dimensional channel systems selected from the PCOD database having a PCOD identifier selected from the group consisting of 8178377, 8285531, 8280370, 8285533, and 8274377.

EMBODIMENT 21 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one or more zeolites with three-dimensional channel systems selected from the PCOD database having a PCOD identifier selected from the group consisting of 8319806, 8311374, 8316340, 8305889, 8065252, 8305207, 8308011, and 8308120.

EMBODIMENT 22 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one of: (a) one or more zeolites with one-dimensional channel systems selected from the group consisting of a CAN-type zeolite, an EUO-type zeolite, a GON-type zeolite, and a VET-type zeolite; or (b) one or more zeolites with three-dimensional channel systems selected from the group consisting of a WEN-type zeolite and an ITH-type zeolite.

EMBODIMENT 23 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one of: (a) one or more zeolites with one-dimensional channel systems including at least one zeolite selected from the PCOD database having a PCOD identifier selected from the group consisting of 8113534, 8296636, 8302206, 8165762, 8302179, 8121102, 8276859, 8149581, 8244356, 8296363, 8321948, 8138688, 8223156, 8245547, 8223914, 8305046, 8152848, 8234126, 8190755, 8127992, 8152156, 8305042, 8137831, 8246562, 8296721, 8258975, 8326325, 8247653, 8165791, 8118469, 8186880, 8203701, 8216070, 8324735, 8318543, 8324823, 8296863, 8304854, 8322003, 8295642, 8205808, 8164336, 8129506, 8322048, 8119337, 8255575, 8159638, 8269679, 8153524, 8319059, 8026125, 8273431, 8295955, 8269459, 8170433, 8164121, 8326255, 8281376, 8301873, 8165656, 8277094, 8233560, 8137616, 8149587, 8169792, 8293991, 8262350, 8264198, 8115891, 8262902, 8306970, 8226024, 8322924, 8152100, 8277433, 8218629, 8054782, 8120251, 8163400, 8247952, 8109723, 8234406, 8216438, 8296333, 8216552, 8150035, 8259735, 8130003, 8137562, 8279697, 8163965, 8324742, 8155533, 8148176, 8072540, 8217078, 8297845, 8165033, 8276974, 8298516, 8054597, 8082795, 8203181, 8326100, 8139317, 8165538, 8326372, 8254444, 8167956, 8054214, 8193268, 8304867, 8223456, 8045653, 8302209, 8219004, 8306237, 8272811, 8157648, 8326099, 8166074, 8158356, 8325576, 8158769, 8121081, 8324818, 8323141, 8247317, 8166112, 8073568, 8277341, 8222738, 8141396, 8232287, 8245510, 8254068, 8152150, 8148869, 8204023, 8326245, 8306845, 8219078, 8164071, 8306066, 8111747, 8204010, 8161093, 8263690, 8205511, 8097894, 8059282, 8329494, 8219685, 8164344, 8311634, 8238847, 8165831, 8134613, 8219214, 8030963, 8141091, 8322611, 8169582, 8282106, 8326097, 8222264, 8030519, 8005350, 8254017, 8114704, 8183709, 8137450, 8274361, 8233586, 8119910, 8264279, 8323852, 8243329, 8254550, 8291455, 8219891, 8308806, 8326257, 8325769, 8004705, 8321111, 8121189, 8147634, 8166054, 8301299, 8279593, 8123558, 8183228, 8281874, 8204430, 8054794, 8124909, 8323165, 8294533, 8148507, 8008796, 8128538, 8233864, 8165171, 8262781, 8231696, 8304734, 8263651, 8076700, 8226706, 8257181, 8222180, 8136633, 8247289, 8228296, 8074110, 8160689, 8245586, 8301408, 8257704, 8281264, 8282839, 8293427, 8246429, 8135370, 8124669, 8152114, 8276300, 8318439, 8304970, 8114386, 8233041, 8311771, 8227536, 8215446, 8271288, 8234171, 8175996, 8311227, 8323124, 8201592, 8159102, 8137417, 8151237, 8160333, 8165896, 8151013, 8289645, 8247005, 8029604, 8222883, 8131207, 8095377, 8175192, 8276346, 8205710, 8279368, 8175541, 8222024, 8326153, 8319790, 8310983, 8122614, 8083868, 8119832, 8216857, 8145217, 8292039, 8221747, 8325766, 8320603, 8323818, 8076935, 8152101, 8277551, 8049792, 8274130, 8165707, 8141266, 8277398, 8183844, 8304796, 8063993, 8046781, 8285996, 8313513, and 8053941; (b) one or more zeolites with two-dimensional channel systems selected from the PCOD database having a PCOD identifier selected from the group consisting of 8178377, 8285531, 8280370, 8285533, and 8274377; or (c) one or more zeolites with three-dimensional channel systems selected from the PCOD database having a PCOD identifier selected from the group consisting of 8319806, 8311374, 8316340, 8305889, 8065252, 8305207, 8308011, and 8308120.

EMBODIMENT 24 can include subject matter (such as an apparatus, a device, a method, or one or more means for performing acts), such as can include a catalyst system for converting at least a fraction of one or more linear or slightly branched alkanes of a hydrocarbon feedstock. The subject matter can include the catalyst system comprising one of: (a) one or more zeolites with one-dimensional channel systems selected from the group consisting of a CAN-type zeolite, an EUO-type zeolite, a GON-type zeolite, and a VET-type zeolite; (b) one or more zeolites with three-dimensional channel systems selected from the group consisting of a WEN-type zeolite and an ITH-type zeolite; (c) one or more zeolites with one-dimensional channel systems including at least one zeolite selected from the PCOD database having a PCOD identifier selected from the group consisting of 8113534, 8296636, 8302206, 8165762, 8302179, 8121102, 8276859, 8149581, 8244356, 8296363, 8321948, 8138688, 8223156, 8245547, 8223914, 8305046, 8152848, 8234126, 8190755, 8127992, 8152156, 8305042, 8137831, 8246562, 8296721, 8258975, 8326325, 8247653, 8165791, 8118469, 8186880, 8203701, 8216070, 8324735, 8318543, 8324823, 8296863, 8304854, 8322003, 8295642, 8205808, 8164336, 8129506, 8322048, 8119337, 8255575, 8159638, 8269679, 8153524, 8319059, 8026125, 8273431, 8295955, 8269459, 8170433, 8164121, 8326255, 8281376, 8301873, 8165656, 8277094, 8233560, 8137616, 8149587, 8169792, 8293991, 8262350, 8264198, 8115891, 8262902, 8306970, 8226024, 8322924, 8152100, 8277433, 8218629, 8054782, 8120251, 8163400, 8247952, 8109723, 8234406, 8216438, 8296333, 8216552, 8150035, 8259735, 8130003, 8137562, 8279697, 8163965, 8324742, 8155533, 8148176, 8072540, 8217078, 8297845, 8165033, 8276974, 8298516, 8054597, 8082795, 8203181, 8326100, 8139317, 8165538, 8326372, 8254444, 8167956, 8054214, 8193268, 8304867, 8223456, 8045653, 8302209, 8219004, 8306237, 8272811, 8157648, 8326099, 8166074, 8158356, 8325576, 8158769, 8121081, 8324818, 8323141, 8247317, 8166112, 8073568, 8277341, 8222738, 8141396, 8232287, 8245510, 8254068, 8152150, 8148869, 8204023, 8326245, 8306845, 8219078, 8164071, 8306066, 8111747, 8204010, 8161093, 8263690, 8205511, 8097894, 8059282, 8329494, 8219685, 8164344, 8311634, 8238847, 8165831, 8134613, 8219214, 8030963, 8141091, 8322611, 8169582, 8282106, 8326097, 8222264, 8030519, 8005350, 8254017, 8114704, 8183709, 8137450, 8274361, 8233586, 8119910, 8264279, 8323852, 8243329, 8254550, 8291455, 8219891, 8308806, 8326257, 8325769, 8004705, 8321111, 8121189, 8147634, 8166054, 8301299, 8279593, 8123558, 8183228, 8281874, 8204430, 8054794, 8124909, 8323165, 8294533, 8148507, 8008796, 8128538, 8233864, 8165171, 8262781, 8231696, 8304734, 8263651, 8076700, 8226706, 8257181, 8222180, 8136633, 8247289, 8228296, 8074110, 8160689, 8245586, 8301408, 8257704, 8281264, 8282839, 8293427, 8246429, 8135370, 8124669, 8152114, 8276300, 8318439, 8304970, 8114386, 8233041, 8311771, 8227536, 8215446, 8271288, 8234171, 8175996, 8311227, 8323124, 8201592, 8159102, 8137417, 8151237, 8160333, 8165896, 8151013, 8289645, 8247005, 8029604, 8222883, 8131207, 8095377, 8175192, 8276346, 8205710, 8279368, 8175541, 8222024, 8326153, 8319790, 8310983, 8122614, 8083868, 8119832, 8216857, 8145217, 8292039, 8221747, 8325766, 8320603, 8323818, 8076935, 8152101, 8277551, 8049792, 8274130, 8165707, 8141266, 8277398, 8183844, 8304796, 8063993, 8046781, 8285996, 8313513, and 8053941; (d) one or more zeolites with two-dimensional channel systems selected from the PCOD database having a PCOD identifier selected from the group consisting of 8178377, 8285531, 8280370, 8285533, and 8274377; or (e) one or more zeolites with three-dimensional channel systems selected from the PCOD database having a PCOD identifier selected from the group consisting of 8319806, 8311374, 8316340, 8305889, 8065252, 8305207, 8308011, and 8308120.

EMBODIMENT 25 can include, or can optionally be combined with, the subject matter of one or any combination of EMBODIMENTS 17-24, to optionally include the channel systems of the zeolite have pore diameters ranging from about 0.45 nanometers to about 0.7 nanometers.

EMBODIMENT 26 can include, or can optionally be combined with, the subject matter of one or any combination of EMBODIMENTS 17-25, to optionally include the catalyst system further comprising a hydrogenation component.

EMBODIMENT 27 can include, or can optionally be combined with, the subject matter of one or any combination of EMBODIMENTS 17-26, to optionally include the hydrogenation component being selected from the group consisting of Group VIII metals, Group VIB metals, and Group IB metals.

EMBODIMENT 28 can include, or can optionally be combined with, the subject matter of one or any combination of EMBODIMENTS 17-27, to optionally include the Group VIII metals comprising platinum, palladium, or mixtures thereof.

EMBODIMENT 29 can include subject matter (such as an apparatus, a device, a method, or one or more means for performing acts), such as can include a method for systematically identifying zeolites suitable to be used in a catalyst system for conversion of at least a fraction of one or more linear or slightly branched alkanes. The subject matter can include evaluating each of a plurality of candidate zeolite structures according to a set of criteria, the set of criteria comprising one or more of: adsorption affinities of linear alkanes with at least 14 carbon atoms, one or more adsorption selectivities of linear over branched alkanes with at least 14 carbon atoms, and one or more adsorption selectivities of linear alkanes of different molecular weights with at least 14 carbon atoms, wherein the adsorption affinities and adsorption selectivities are computed for one or more state points representing conditions of the conversion.

EMBODIMENT 30 can include, or can optionally be combined with, the subject matter of EMBODIMENT 29, to optionally include the adsorption affinity of the linear alkanes, the adsorption selectivity of the linear over branched alkanes, and the adsorption selectivity of the linear alkanes of different molecular weights are evaluated at hydroisomerization reaction conditions including the equilibrium loading of n-octadecane, Q_{nC18}, S_{B3}, and S_{L3}, wherein S_{B3} is defined as:

$${S}_{B 3} = \frac{3 {Q}_{nC 18}}{{Q}_{2 mC 17} + {Q}_{4 mC 17} + {Q}_{22 d mC 16}} ,$$

and S_{L3} is defined as:

$${S}_{L 3} = \frac{\sqrt{{Q}_{nC 24} {Q}_{nC 30}}}{{Q}_{nC 18}}$$

wherein Q_{2mC17}, Q_{4mC17}, Q_{22dmC16}, Q_{nC24}, and Q_{nC30} denote the equilibrium loadings of 2-methyl- and 4-methylheptadecane, 2,2-dimethylhexadecane, n-tetracosane, and n-triacontane, respectively.

EMBODIMENT 31 can include, or can optionally be combined with, the subject matter of one or any combination of EMBODIMENTS 29 or 30, to optionally include the set of criteria maximizing P=Q_{nC18}×S_{B3}/S_{L3}.

EMBODIMENT 32 can include, or can optionally be combined with, the subject matter of one or any combination of EMBODIMENTS 29-31, to optionally include the group of candidate zeolite structures being selected from all experimentally synthesized zeolites.

EMBODIMENT 33 can include, or can optionally be combined with, the subject matter of one or any combination of EMBODIMENTS 29-32, to optionally include the group of candidate zeolite structures being selected from a database of predicted zeolites.

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a molding system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented, at least in part. Some examples can include a computer-readable medium or machine-readable medium encoded with instructions operable to configure an electronic device to perform methods or method steps as described in the above examples. An implementation of such methods or method steps can include code, such as microcode, assembly language code, a higher-level language code, or the like. Such code can include computer readable instructions for performing various methods. The code may form portions of computer program products. The code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The Abstract is provided to allow the reader to quickly ascertain the nature of the technical disclosure. It is submitted with the understanding that it will not be used to interpret or limit the scope or meaning of the claims.

Although the invention has been described with reference to exemplary embodiments, workers skilled in the art will recognize that changes may be made in form and detail without departing from the spirit and scope of the invention.

1. A process for conversion of at least a fraction of one or more linear or slightly branched alkanes of a hydrocarbon feedstock, the process comprising contacting the hydrocarbon feedstock with a catalyst system comprising one or more zeolites selected from either:
one or more zeolites with having one-dimensional channel systems selected from the group consisting of: a CAN-type zeolite, a GON-type zeolite, or a VET-type zeolite; or
one or more zeolites having three-dimensional channel systems selected from the group consisting of: a WEN-type zeolite or an ITH-type zeolite.

2. (canceled)

3. A process for conversion of at least a fraction of one or more linear or slightly branched alkanes of a hydrocarbon feedstock, the process comprising contacting the hydrocarbon feedstock with a catalyst system, the catalyst system comprising;
one or more zeolites with one-dimensional channel systems including at least one zeolite selected from the PCOD database having a PCOD identifier selected from the group consisting of: 8113534, 8296636, 8302206, 8165762, 8302179, 8121102, 8276859, 8149581, 8244356, 8296363, 8321948, 8138688, 8223156, 8245547, 8223914, 8305046, 8152848, 8234126, 8190755, 8127992, 8152156, 8305042, 8137831, 8246562, 8296721, 8258975, 8326325, 8247653, 8165791, 8118469, 8186880, 8203701, 8216070, 8324735, 8318543, 8324823, 8296863, 8304854, 8322003, 8295642, 8205808, 8164336, 8129506, 8322048, 8119337, 8255575, 8159638, 8269679, 8153524, 8319059, 8026125, 8273431, 8295955, 8269459, 8170433, 8164121, 8326255, 8281376, 8301873, 8165656, 8277094, 8233560, 8137616, 8149587, 8169792, 8293991, 8262350, 8264198, 8115891, 8262902, 8306970, 8226024, 8322924, 8152100, 8277433, 8218629, 8054782, 8120251, 8163400, 8247952, 8109723, 8234406, 8216438, 8296333, 8216552, 8150035, 8259735, 8130003, 8137562, 8279697, 8163965, 8324742, 8155533, 8148176, 8072540, 8217078, 8297845, 8165033, 8276974, 8298516, 8054597, 8082795, 8203181, 8326100, 8139317, 8165538, 8326372, 8254444, 8167956, 8054214, 8193268, 8304867, 8223456, 8045653, 8302209, 8219004, 8306237, 8272811, 8157648, 8326099, 8166074, 8158356, 8325576, 8158769, 8121081, 8324818, 8323141, 8247317, 8166112, 8073568, 8277341, 8222738, 8141396, 8232287, 8245510, 8254068, 8152150, 8148869, 8204023, 8326245, 8306845, 8219078, 8164071, 8306066, 8111747, 8204010, 8161093, 8263690, 8205511, 8097894, 8059282, 8329494, 8219685, 8164344, 8311634, 8238847, 8165831, 8134613, 8219214, 8030963, 8141091, 8322611, 8169582, 8282106, 8326097, 8222264, 8030519, 8005350, 8254017, 8114704, 8183709, 8137450, 8274361, 8233586, 8119910, 8264279, 8323852, 8243329, 8254550, 8291455, 8219891, 8308806, 8326257, 8325769, 8004705, 8321111, 8121189, 8147634, 8166054, 8301299, 8279593, 8123558, 8183228, 8281874, 8204430, 8054794, 8124909, 8323165, 8294533, 8148507, 8008796, 8128538, 8233864, 8165171, 8262781, 8231696, 8304734, 8263651, 8076700, 8226706, 8257181, 8222180, 8136633, 8247289, 8228296, 8074110, 8160689, 8245586, 8301408, 8257704, 8281264, 8282839, 8293427, 8246429, 8135370, 8124669, 8152114, 8276300, 8318439, 8304970, 8114386, 8233041, 8311771, 8227536, 8215446, 8271288, 8234171, 8175996, 8311227, 8323124, 8201592, 8159102, 8137417, 8151237, 8160333, 8165896, 8151013, 8289645, 8247005, 8029604, 8222883, 8131207, 8095377, 8175192, 8276346, 8205710, 8279368, 8175541, 8222024, 8326153, 8319790, 8310983, 8122614, 8083868, 8119832, 8216857, 8145217, 8292039, 8221747, 8325766, 8320603, 8323818, 8076935, 8152101, 8277551, 8049792, 8274130, 8165707, 8141266, 8277398, 8183844, 8304796, 8063993, 8046781, 8285996, 8313513, or 8053941.

4. A process for conversion of at least a fraction of one or more linear or slightly branched alkanes of a hydrocarbon feedstock, the process comprising contacting the hydrocarbon feedstock with a catalyst system comprising one or more zeolites having a two-dimensional channel systems selected from the PCOD database having a PCOD identifier selected from the group consisting of: 8178377, 8285531, 8280370, 8285533, or 8274377.

5. A process for conversion of at least a fraction of one or more linear or slightly branched alkanes of a hydrocarbon feedstock, the process comprising contacting the hydrocarbon feedstock with a catalyst system comprising one or more zeolites, the catalyst system comprising one or more zeolites with three-dimensional channel systems selected from the PCOD database having a PCOD identifier selected from the group consisting of: 8319806, 8311374, 8316340, 8305889, 8065252, 8305207, 8308011, or 8308120.

6. (canceled)

7. The process of claim 1, wherein the catalyst system further comprises a hydrogenation component.

8. The process of claim 7, wherein the hydrogenation component comprises at least one of: one or more Group VIII metals, one or more Group VIB metals, and one or more Group D3 metals.

9. The process of claim 8, wherein the Group VIII metals comprise Pt, Pd, or mixtures thereof.

10. The process of claim 1, wherein the contacting is performed at hydroisomerization conditions with added hydrogen, wherein the hydroisomerization conditions include one or more of:
a temperature of 473-773 K;
a pressure of 0.1-20 MPa; and
a liquid hourly space velocity of 0.1-20 hr⁻¹.

11-27. (canceled)

28. The process of claim 3, wherein the catalyst system further comprises a hydrogenation component.

29. The process of claim 28, wherein the hydrogenation component comprises at least one of: one or more Group VIII metals, one or more Group VIB metals, and one or more Group D3 metals.

30. The process of claim 29, wherein the Group VIII metals comprise Pt, Pd, or mixtures thereof.

31. The process of claim 3, wherein the contacting is performed at hydroisomerization conditions with added hydrogen, wherein the hydroisomerization conditions include one or more of:
a temperature of 473-773 K;
a pressure of 0.1-20 MPa; and
a liquid hourly space velocity of 0.1-20 hr⁻¹.

32. The process of claim 4, wherein the catalyst system further comprises a hydrogenation component.

33. The process of claim 32, wherein the hydrogenation component comprises at least one of: one or more Group VIII metals, one or more Group VIB metals, and one or more Group D3 metals.

34. The process of claim 33, wherein the Group VIII metals comprise Pt, Pd, or mixtures thereof.

35. The process of claim 5, wherein the contacting is performed at hydroisomerization conditions with added hydrogen, wherein the hydroisomerization conditions include one or more of:
a temperature of 473-773 K;
a pressure of 0.1-20 MPa; and
a liquid hourly space velocity of 0.1-20 hr⁻¹.

36. The process of claim 4, wherein the catalyst system further comprises a hydrogenation component.

37. The process of claim 36, wherein the hydrogenation component comprises at least one of: one or more Group VIII metals, one or more Group VIB metals, and one or more Group D3 metals.

38. The process of claim 37, wherein the Group VIII metals comprise Pt, Pd, or mixtures thereof.

39. The process of claim 5, wherein the contacting is performed at hydroisomerization conditions with added hydrogen, wherein the hydroisomerization conditions include one or more of:
a temperature of 473-773 K;
a pressure of 0.1-20 MPa; and
a liquid hourly space velocity of 0.1-20 hr⁻¹.
